(19) United States
(12) Patent Application Publication
(10) Pub. No.: US 2006/0199237 A1
(43) Pub. Date: Sep. 7, 2006

(54) Clinical assay for monitoring of agmatine

(76) Inventors: Daniel J. Smith, Stow, OH (US); Eugenio H. Zabaleta, Mansfield, OH (US); Murray Rosenthal, Toms River, NJ (US)

Correspondence Address: ROETZEL AND ANDRESS, 222 SOUTH MAIN STREET, AKRON, OH, 44308, US

(21) Appl. No.: 11/366,097

(22) Filed: Mar. 2, 2006

Related U.S. Application Data
(60) Provisional application No. 60/658,048, filed on Mar. 2, 2005.

(51) Int. Cl. G01N 33/537; G01N 33/53; G01N 33/543

(52) U.S. Cl. 435/7.92

(57) ABSTRACT

The present invention is directed to a process for determining the level of agmatine in biological samples, especially that of human subjects. In one embodiment, the present invention comprises an assay and a method for quantitatively detecting agmatine in blood serum and other body fluids.

### RELATED APPLICATION DATA

This application claims priority to previously filed U.S. Provisional Patent Application No. 60/658,048, which is hereby incorporated by reference it its entirety.

### FIELD OF THE INVENTION

The present invention is directed to a process for determining the level of agmatine in biological samples, especially that of human subjects. In one embodiment, the present invention comprises an assay and a method for quantitatively detecting agmatine in blood serum and other body fluids.

### BACKGROUND OF THE INVENTION

Agmatine is used in treating and diagnosing a variety of disorders. Monitoring agmatine levels in patients undergoing agmatine treatments is especially important due to its toxicity. At present, the only methods available for agmatine determination are not suitable for routine clinical laboratory practice because they require expensive instrumentation and highly skilled technicians. Common practice is to measure agmatine levels using gas chromatography/mass spectrometry (GC-MS) or a high pressure liquid chromatography (HPLC) method in combination with a fluorescence detector. This test requires the use of skilled clinicians to determine the serum level of agmatine in a patient.

Agmatine has been used as a treatment for neuropathic pain, which is the pain produced due to a dysfunction or damage of the central or peripheral nervous system. Some diseases, such as cancer or AIDS, have a state with neuropathic pain. Inflammatory conditions such as arthritis present this kind of pain also, as do some metabolic disorders (e.g., diabetes). Such pain can also occur after injuries or amputations.

Biological activity of agmatine that has been discovered in other areas. In the kidneys, agmatine increases the excretion of Na+independent of the blood flow; this produces a diuretic effect. Agmatine also has an effect on Luteinizing Hormone by releasing LHRH (Luteinizing Hormone Releasing Hormone) from the Hypothalamus. Agmatine intensifies morphine analgesia due to an adrenergic receptor. It also attenuates the effect of morphine abstinence syndrome in morphine-addicted rats. It has vasodilatory and hypotensor effects on the vascular system, as well as inhibiting nitric oxide synthase in inflammatory processes. In isolated adipocytes, it has an insulin-like effect and results in the release of insulin in a dose dependent manner from islet cells. In patients with depression, the agmatine level in plasma was found to be significantly elevated. Some evidence suggests that agmatine could meet the criteria to be considered a neurotransmitter. Agmatine levels in human plasma or serum may increase in neurological diseases such as Epilepsy, Seizures, Alzheimer, Bipolar Disorders, Parkinson, Manic-depression, Cushing Syndrome, dementia, dementia with Lewy Bodies, Diabetic neuropathy, Dystonia, Huntington Disease, Meningitis, Stroke, Spinal Cord Injuries, Brain Tumors, Spinal Muscular Atrophy and Botulism. As the foregoing suggests, serum agmatine level determination is useful in the treatment or diagnosis of a wide variety of conditions.

### SUMMARY OF THE INVENTION

In one embodiment, the present invention relates to a process for agmatine determination. More particularly the present invention is directed to such a process, which requires less equipment and training than current processes and is a significant step forward in the biomedical arts. More particularly, the present invention is directed to an enzyme-linked immunosorbent assay (ELISA) using an anti-agmatine antibody to determine the concentration of agmatine in serum, plasma and other body fluids, especially those of a human.

In one embodiment, the present invention relates to a clinical assay that uses the steps of: (1) exposing at least one ELISA surface to agmatine such that the agmatine binds to it resulting in an agmatine-modified surface; (2) introducing an agmatine-containing sample to the agmatine-modified surface so as to form a surface-agmatine plus free-agmatine system; (3) adding an anti-agmatine antibody to the system so that surface-agmatine and free-agmatine must compete to bind to the antibody; (4) washing the surface such that substantially only surface bound moieties remain; (5) reacting any surface-bound anti-agmatine antibodies with an immunoglobin G (IgG), wherein the IgG is itself bound to an enzyme; (6) washing the surface such that substantially only surface bound moieties remain; and (7) treating any surface-bound enzyme with a substrate, where the enzyme's reaction with the substrate is detectable by a means selected from the group consisting of visual inspection, visible fluorescence/phosphorescence spectroscopy, and absorbance spectroscopy in the ultraviolet (UV), visible (Vis) or near infrared (NIR) regions of the spectrum.

In another embodiment, the present invention relates to a clinical assay comprising the steps of: (a) saturating at least one enzyme-linked immunosorbent assay surface with agmatine such that the agmatine binds thereto resulting in an agmatine-modified surface; (b) introducing an agmatine-containing sample to the agmatine-modified surface so as to form a surface-agmatine free-agmatine system; (c) adding an anti-agmatine antibody to the system so that surface-agmatine and free-agmatine must compete to bind to the antibody, wherein the antibody is bound to an enzyme; (d) washing the surface such that substantially only surface bound moieties remain; and (e) treating any surface-bound enzyme with a substrate wherein the enzyme's reaction with the substrate is detectable by a means selected from the group consisting of visual, emission, UV-Vis absorbance, NIR absorbance, and IR absorbance.

In still another embodiment, the present invention relates to a clinical assay comprising the steps of: (i) introducing an agmatine-containing sample to at least one enzyme-linked immunoassay surface such that the agmatine binds substantially quantitatively thereto resulting in an agmatine-modified surface; (ii) washing the surface such that substantially only surface bound moieties remain; (iii) adding an excess of an anti-agmatine antibody to the surface such that it may bind substantially all of the surface-bound agmatine, wherein the antibody is bound to an enzyme; (iv) washing the surface such that substantially only surface bound moieties remain; and (v) treating any surface-bound enzyme with a substrate wherein the enzyme's reaction with the substrate is detectable by a means selected from the group consisting of visual, emission, UV-Vis absorbance, NIR absorbance, and IR absorbance.

The following terms are specially defined. The term clinical assay, as used herein means a process or method for analysis in a clinical laboratory environment.

### BRIEF DESCRIPTION OF THE DRAWINGS

FIG. 1 is a symbolic representation of the agmatine reaction with the anti-agmatine antibody in a container having an ELISA surface;

FIG. 2 is a symbolic representation of the Agmatine+anti-agmatine antibody conjugate reaction with the immunoglobin G antibody+enzyme conjugate; and

FIG. 3 is a symbolic representation of the development step using a substrate including substrate A being converted to product B by an enzyme tethered to immunoglobin G (IgG).

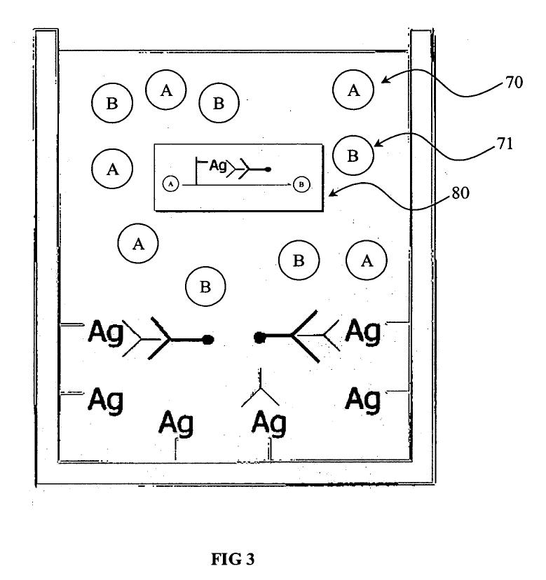

### DETAILED DESCRIPTION OF THE INVENTION

The present invention is directed to a process for determining the level of agmatine in biological samples, especially that of human subjects. In one embodiment, the present invention comprises an assay and a method for quantitatively detecting agmatine in blood serum and other body fluids.

In one embodiment, the present invention involves the steps of: (1) introducing an agmatine-containing sample to at least one enzyme-linked immunoassay surface such that the agmatine binds substantially quantitatively thereto resulting in an agmatine-modified surface; (2) washing the surface such that substantially only surface bound moieties remain; (3) adding an excess of an anti-agmatine antibody to the surface such that it may bind substantially all of the surface-bound agmatine; (4) reacting any surface-bound anti-agmatine antibodies with an IgG, wherein the IgG is itself bound to an enzyme; (5) washing the surface such that substantially only surface bound moieties remain; and (6) treating any surface-bound enzyme with a substrate, wherein the enzyme's reaction with the substrate is detectable by a means selected from the group consisting of visual, emission, UV-Vis absorbance, NIR absorbance, and IR absorbance.

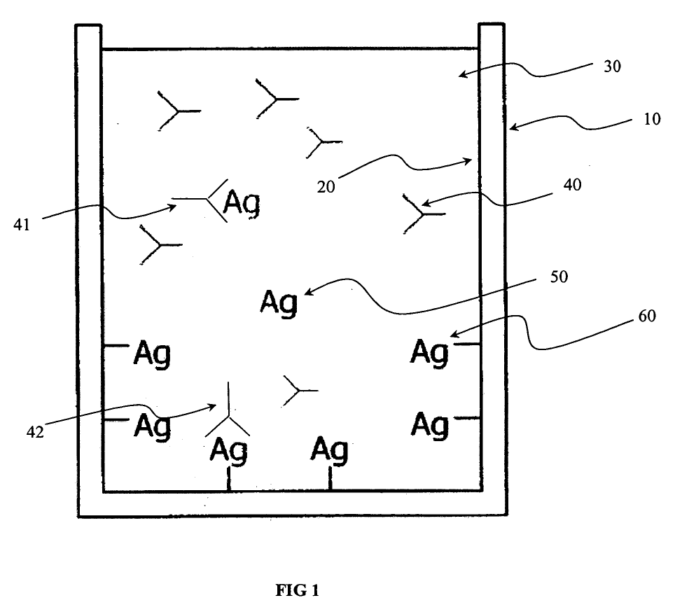

More particularly, the process of the present invention comprises treating at least one enzyme-linked immunosorbent assay (ELISA) surface with agmatine 60 so that the agmatine 60 is tethered to the surface more or less permanently, as shown in FIG. 1. The foregoing ELISA surface(s) 20 are usually located in a reaction well 10 or similar structure. Then the surface is washed free of unbound agmatine. Generally, the ELISA surface would now be “blocked”, meaning it would be treated in such a way as to prevent the further binding of agmatine or any other substance that may interfere with test results.

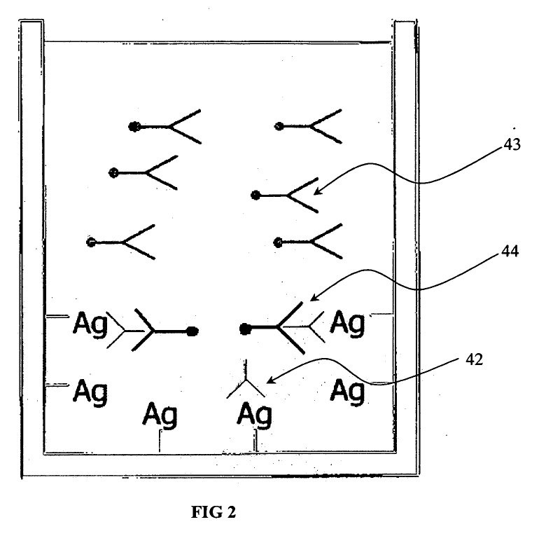

Then, and with reference to FIG. 1, a solution 30 comprising either an agmatine standard or a body fluid sample is introduced to the surface. A first antibody 40 (an anti-agmatine antibody) is added to the ELISA surface(s) 20 involved in the present process including those in contact with standards and those in contact with samples. This “first” antibody 40 is specially chosen to bind selectively to either free agmatine 50 or surface-bound agmatine 60, to form conjugates 41 that are free in solution and conjugates 42 bound to the ELISA surface (in any case the antibody may be either polyclonal or monoclonal). Therefore, surface agmatine 60 and free agmatine 50 must compete for antibody 40. Thus, the amount of antibody 40 coupling to surface-bound agmatine 60 is attenuated by the amount of free agmatine 50. When the foregoing coupling reaction is complete the surface is washed free of any loose agmatine 50 and loose agmatine-antibody conjugates 41. At this point an enzyme-labeled immunoglobulin G (IgG) 43 is added to the ELISA surface, and allowed to bind with any ELISA surface-bound agmatine-antibody conjugates 44 as shown in FIG. 2.

Next, the ELISA surface is washed again to remove excess enzyme-linked IgG 43, and then it is treated with a substrate 70 upon which the enzyme linked to the IgG 43 will react 80 to form a product 71. The product 71 causes a spectroscopically detectable change in the liquid over the ELISA surface; for instance, a change in its UV-Vis absorption spectrum. The concentration of agmatine in a serum sample processed according to the foregoing method can be determined by comparing the signal resulting from the serum sample to a calibration curve generated by running a series of known agmatine standards. Alternatively, the presence of agmatine can be detected qualitatively by comparison to an appropriate control or blank.

In general the present invention is directed to a process for quantitatively detecting agmatine in blood serum. In one embodiment, the present invention comprises seven main steps: (a) coating an enzyme-linked immunosorbent assay (ELISA) plate with agmatine; (b) blocking the ELISA plate so that no further agmatine may bind to the surface; (c) exposing the agmatine coated plate to an analyte body fluid or an agmatine standard; (d) introducing an anti-agmatine antibody to the sample or standard while it is in contact with the agmatine coated ELISA plate; (e) binding any agmatine+antibody conjugates to an enzyme-labeled immunoglobulin G; (f) exposing any agmatine+antibody+enzyme-labeled immunoglobulin G conjugates to a substrate of the enzyme wherein the reaction of the enzyme and substrate produces a spectroscopically detectable result; and (g) measuring any spectroscopically detectable result of the enzyme/substrate reaction.

Measurements obtained by the process of the present invention are used in one of two ways. The measurement can either be used to construct a calibration curve (analytical signal versus concentration), or it can be compared to such a calibration curve to determine an unknown concentration. Additionally, the foregoing steps can be grouped into four parts: (I) ELISA plate coating; (II) competitive immunoassay; (III) development; and (IV) detection.

One illustrative embodiment comprises an analyte having 0 ng/ml to 100 ng/ml agmatine, which is the range typically found in human plasma. An illustrative embodiment further comprises a 1:2000 dilution of the first antibody (i.e., the anti-agmatine antibody), and 1:250 dilution of the second antibody (i.e., the IgG) and a 5 μl/ml solution of agmatine to coat the plate.

One illustrative embodiment comprises the following:

Part I: ELISA Plate Coating:

(1) Put 200 μL of a 5 μg/mL aqueous agmatine sulfate solution in each ELISA plate well; 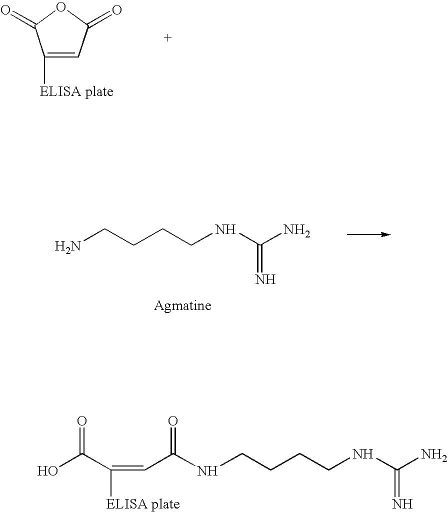
(2) Incubate for one hour at room temperature;
(3) Wash 3 times with 200 μL of a wash solution; do not discharge the last wash. The solution comprises: 0.3 grams nonfat dry milk (NFDM), 0.05 grams of polyoxyethylene sorbitan monolaurate 70% in water, and 100 mL of phosphate-buffered saline (PBS), having a pH 7.4. The PBS solution comprises: 7.650 grams of NaCl, 1.243 grams of Na₂HPO₄.7H₂O, 0.210 grams of KH₂PO₄ in 1 liter of distilled water;
(4) Incubate for one hour at room temperature; and
(5) Rinse each well with 300 μL of PBS.
   Part II: Competitive Immunoassay:
(1) Put 100 μL of the sample (e.g., human plasma) into a well. Put the same amount of an agmatine standard(s) into another well(s). Agmatine standards are prepared from stock solution (1 mg/ml of agmatine in distilled water) with a solution of 0.3% nonfat dried milk;
(2) Add 100 μL of anti-agmatine antibody to each well (1:2000 dilution) (see FIG. 1);
(3) Incubate for one hour at room temperature;
(4) Wash three times with 200 μL of wash solution;
(5) Add 200 μL of IgG affinity purified, horseradish peroxidase conjugated, antibody; 1:250 dilution in wash solution (see FIG. 2);
(6) Incubate for one hour at room temperature;
(7) Wash three times with 200 μL of wash solution; and
(8) Wash one time with 200 μL of PBS.
   Part III and IV: Development and Detection
(1) Add 200 μL of the substrate solution to each well after a PBS wash (the substrate solution comprises 0.4 mg/ml OPD, 0.4 mg/ml urea hydrogen peroxide, and 0.05 M phosphate-citrate, pH 5.0);
(2) Incubate for 30 minutes in the dark;
(3) Add 50 μL of 3M H₂SO₄;
(4) Read absorption at 492 nm;
(5) Compare to calibration curve to determine concentration, or compare to control or blank to qualitatively determine the presence or absence of agmatine.

The foregoing illustrative embodiment is, in one embodiment, carried out with several agmatine standards of various agmatine concentrations so as to formulate a calibration curve to which samples of unknown agmatine concentration may be compared.

In another embodiment nonfat dry milk (NFDM) is replaced by N-2-hydroxyehtylpiperazine-N′-2-ethanesulfonic acid (HEPES), which provides better reagent stability. In still another embodiment of the present invention, the horseradish peroxidase is conjugated directly to the anti-agmatine antibody rather than an IgG, which eliminates the need for the extra IgG binding step. This can make the assay process easier, shorter and more reproducible by decreasing the possibility of human error. In still another embodiment, the substrate of the development reaction is 2,2′-azino-bis-(3-ethylbenzthiazoline-6-sulfonic acid (ABTS) rather than o-phenylenediamine dihydrochloride (OPD). ABST provides further process simplicity and is less costly than OPD.

In still another embodiment of the present invention samples are introduced to a bare ELISA plate so that the agmatine in the sample is permitted to bind directly to the ELISA plate. The sample of this embodiment is diluted so that the reactive sites on the ELISA plate are in sufficient excess relative the amount of agmatine in solution so that the reaction immobilizing the agmatine to the plate surface is substantially quantitative. The plate is then washed and reacted with an anti-agmatine antibody. The antibody may or may not be tethered to an enzyme. If it is so tethered, then the next step is to react the enzyme with a substrate, the product of which is optically, spectroscopically, or otherwise detectable. If the antibody is not tethered to an enzyme, then the next step is to react the agmatine+anti-agmatine antibody conjugate with an IgG, which is tethered to such an enzyme and develop the assay accordingly. The amount of agmatine in the sample is determined by comparing the signal derived therefrom to a calibration curve composed of signals from known standard agmatine concentrations.

Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents.

What is claimed is:

1. A clinical assay process comprising the steps of:
(1) binding a quantity of agmatine to at least one enzyme-linked immunosorbent assay surface resulting in an agmatine-modified surface;
(2) treating the surface so that no further agmatine may bind thereto; Introducing an agmatine-containing sample to the agmatine-modified surface so as to form a surface-agmatine free-agmatine system;
(3) adding an anti-agmatine antibody to the system so that surface-agmatine and free-agmatine must compete to bind to the antibody;
(4) binding any surface-bound anti-agmatine antibodies to an immunoglobin G, wherein the immunoglobin G is also bound to an enzyme;
(5) treating surface-bound enzyme with a substrate wherein the enzyme's reaction with the substrate is spectroscopically detectable; and
(6) measuring a spectroscopically detectable result relative to suitable control or calibration curve.

2. The process of claim 1, wherein the spectroscopically detectable result is measured by a method selected from the group consisting of visible emission spectroscopy, UV-Vis absorption spectroscopy, NIR absorption spectroscopy, and IR absorption spectroscopy.

3. A clinical assay process comprising the steps of:
(a) introducing an agmatine-containing sample to at least one enzyme-linked immunoassay surface such that the agmatine binds substantially quantitatively thereto resulting in an agmatine-modified surface;
(b) treating the surface so that no further agmatine may bind thereto;
(c) adding an excess of an anti-agmatine antibody to the surface such that it may bind substantially all of the surface-bound agmatine;
(d) reacting any surface-bound anti-agmatine antibodies with an immunoglobin G, wherein the immunoglobin G is also bound to an enzyme;
(e) treating any surface-bound enzyme with a substrate wherein the enzyme's reaction with the substrate is spectroscopically detectable; and
(f) measuring a spectroscopically detectable result relative to suitable control or calibration curve.

4. The process of claim 3, wherein the spectroscopically detectable result is measured by a method selected from the group-consisting of visible emission spectroscopy, UV-Vis absorption spectroscopy, NIR absorption spectroscopy, and IR absorption spectroscopy.

5. A clinical assay process comprising the steps of:
(i) binding a quantity of agmatine to at least one enzyme-linked immunosorbent assay surface resulting in an agmatine-modified surface;
(ii) treating the surface so that no further agmatine may bind thereto;
(iii) introducing an agmatine-containing sample to the agmatine-modified surface so as to form a surface-agmatine free-agmatine system;
(iv) adding an anti-agmatine antibody to the system so that surface-agmatine and free-agmatine must compete to bind to the antibody, wherein the anti-agmatine antibody is also bound to an enzyme;
(v) treating any surface-bound enzyme with a substrate wherein the enzyme's reaction with the substrate is spectroscopically detectable; and
(vi) measuring a spectroscopically detectable result relative to suitable control or calibration curve.

6. The process of claim 5, wherein the spectroscopically detectable result is measured by a method selected from the group consisting of visible emission spectroscopy, UV-Vis absorption spectroscopy, NIR absorption spectroscopy, and IR absorption spectroscopy.

7. A clinical assay process comprising the steps of:
(A) introducing an agmatine-containing sample to at least one enzyme-linked immunoassay surface such that the agmatine binds substantially quantitatively thereto resulting in an agmatine-modified surface;
(B) adding an excess of an anti-agmatine antibody to the surface such that it may bind substantially all of the surface-bound agmatine, wherein the anti-agmatine antibody is also bound to an enzyme;
(C) treating surface-bound enzyme with a substrate wherein the enzyme's reaction with the substrate is spectroscopically detectable; and
(D) measuring a spectroscopically detectable result relative to suitable control or calibration curve.

8. The process of claim 7, wherein the spectroscopically detectable result is measured by a method selected from the group consisting of visible emission spectroscopy, UV-Vis absorption spectroscopy, NIR absorption spectroscopy, and IR absorption spectroscopy.

9. An agmatine assay kit comprising:
an anti-agmatine antibody solution;
an immunoglobin antibody solution, wherein the immunoglobin is conjugated to an enzyme; and
a substrate solution.

10. The kit of claim 9, further comprising at least one enzyme-linked immunosorbent assay surface.

11. The kit of claim 10, comprising at least one enzyme-linked immunosorbent assay plate.

12. The kit of claim 9, further comprising at least one wash buffer.

13. The kit of claim 12, wherein the at least one wash buffer is selected from the group consisting of phosphate buffered saline, and non-fat dry milk solution.

14. The kit of claim 13, wherein the phosphate buffered saline solution comprises an aliquot of a solution made from the following components 7.650 grams of NaCl, 1.243 grams of Na₂HPO₄.7H₂O, 0.210 grams KH₂PO₄ in 1 liter of distilled water.

15. The kit of claim 14, wherein the non-fat dry milk solution comprises an aliquot of a solution made from the following ingredients 0.3 grams nonfat dry milk, 0.05 grams of polyoxyethylene sorbitan monolaurate 70% in water, and 100 mL of phosphate-buffered saline having a pH 7.4.

16. The kit of claim 9, further comprising at least one agmatine standard.
